(19) United States
(12) Patent Application Publication
(10) Pub. No.: US 2022/0307004 A1
(43) Pub. Date: Sep. 29, 2022

(54) Multi-Enzyme Nanoparticle-Assisted Stable Isotope Incorporation Into Small Molecules by Channeling

(71) Applicant: The Government of the United States of America, as represented by the Secretary of the Navy, Arlington, VA (US)

(72) Inventors: Igor L. Medintz, Springfield, VA (US); Joyce C. Breger, Washington, DC (US); Gregory A. Ellis, Washington, DC (US); Kirk Malone, Manchester (GB); Nigel Scrutton, Manchester (GB)

(21) Appl. No.: 17/705,781

(22) Filed: Mar. 28, 2022

Related U.S. Application Data
(60) Provisional application No. 63/167,142, filed on Mar. 29, 2021.

(51) Int. Cl. C12N 11/18; C07B 59/00; C12N 9/04

(52) U.S. Cl. CPC C12N 11/18; C07B 59/002; C07B 59/001; C12N 9/0006; C07B 2200/05; C12Y 101/01162; C12Y 114/13022; C12Y 403/01024; C12Y 101/01002

(57) ABSTRACT

Multi-enzyme systems attached to nanoparticles are effective to efficiently and controllably incorporate stable isotopes (such as deuterium) during the synthesis of small molecules. In one example, deuterium is incorporated into (+)-dihydrocarvide using a cascade involving the enzymes (a) pentaerythritol tetranitrate reductase (PETNR) and (b) flavin-dependent cyclohexanone monooxygenase triple variant F249A/F280A/F435A (CHMO_{3M}).

### CROSS-REFERENCE TO RELATED APPLICATIONS

This application claims the benefit of U.S. Provisional Patent Application No. 63/167,142 filed on Mar. 29, 2022, the entirety of which is incorporated herein by reference.

### FEDERALLY-SPONSORED RESEARCH AND DEVELOPMENT

The United States Government has certain ownership rights in this invention.

Licensing inquiries may be directed to Office of Technology Transfer, US Naval Research Laboratory, Code 1004, Washington, DC 20375, USA; +1.202.767.7230; techtran@nrl.navy.mil, referencing NC 112,742.

### BACKGROUND

Several research areas including medicine and organic chemistry rely on the preparation of small molecules incorporating isotopic labels.

For example, in medical applications once might wish to label and follow a drug candidate to determine how it is metabolized and confirm its mechanism of action. Furthermore, because the carbon-deuterium bond is stronger than a conventional carbon-hydrogen bond, drugs that incorporate deuterium can exhibit better half-lives, better bioavailability, better metabolic profiles, and better safety, while retaining therapeutic ability. On example is AUSTEDO (deutetrabenazine), used to treat for chorea from Huntington's Disease and the first deuterated drug to receive approval from the United States Food and Drug Administration. The drug is reported to have fewer side effects than the non-deuterated form of the drug.

A need exists for techniques for the efficient incorporation of isotopes into small molecules.

### BRIEF SUMMARY

As described herein, multi-enzyme systems attached to nanoparticles are effective to efficiently and controllably incorporate stable isotopes (such as deuterium) during the synthesis of small molecules.

In one embodiment, a method of incorporating a radioisotope into a product molecule includes providing a nanoparticle attached to a plurality of enzymes configured as an enzymatic cascade such that a product of a first enzyme is a substrate of the second enzyme and so forth; providing a radioisotope source and a source substrate to the enzymatic cascade; and allowing the enzymatic cascade to act on the radioisotope source and the source substrate, thereby transferring a radioisotope from the radioisotope source into a product molecule.

In various aspects, a radioisotope source can be provided in the form of a solvent (such as deuterated water) and/or one or more cofactors of the enzymes.

In certain aspects, the radioisotope source is a deuterium source such as deuterated water, deuterated nicotinamide adenine dinucleotide phosphate (NADPH-²H), deuterated reduced nicotinamide adenine dinucleotide (NADH-²H), or a combination thereof.

In further aspects, the enzymatic cascade can include one or more reductase enzymes effective to utilize at least one of the radioisotope sources. Example of such reductases include pentaerythritol tetranitrate reductase (PETNR) or carboxylic acid reductase (CAR).

One exemplary embodiment is a method of incorporating deuterium into (+)-dihydrocarvide includes providing a nanoparticle attached to two enzymes, namely (a) pentaerythritol tetranitrate reductase (PETNR) and (b) flavin-dependent cyclohexanone monooxygenase (CHMO); contacting the nanoparticle with carvone and one or more deuterium sources; and allowing the enzymes to act on the carvone and the one or more deuterium sources, thereby producing deuterated dihydrocarvide, wherein the one or more deuterium sources comprise deuterated water. deuterated nicotinamide adenine dinucleotide phosphate, or a combination thereof.

Another exemplary embodiment is a method of incorporating deuterium into cinnamyl alcohol including providing a nanoparticle attached to three enzymes, namely (a) phenylalanine ammonia lyase (PAL), (b) carboxylic acid reductase (CAR), and (c) alcohol dehydrogenase (ADH); contacting the nanoparticle with phenylalanine and one or more deuterium sources; and allowing the enzymes to act on the phenylalanine and the one or more deuterium sources, thereby producing deuterated cinnamyl alcohol, wherein the one or more deuterium sources comprise deuterated nicotinamide adenine dinucleotide phosphate, deuterated reduced nicotinamide adenine dinucleotide, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, and 1C illustrate the incorporation of deuterium into (+)-dihydrocarvide.

FIGS. 2A and 2B show incorporation of deuterium into cinnamaldhyde and cinnamyl alcohol.

FIGS. 3A-3C provide gas chromatography/mass spectroscopy (GC/MS) spectra indicating PETNR can incorporate one deuterium from ²H₂O and one deuterium from NADPH-²H into dihydrocarvone product and can incorporate deuterium in the presence of QD.

FIGS. 4A-4D are GC/MS spectra indicating PETNR and CHMO_{3M} can incorporate one deuterium from ²H₂O and one deuterium from NADPH-²H into dihydrocarvide product, and can incorporate deuterium in the presence of QD.

FIGS. 5A-5D provide GC/MS spectra indicating KRED can incorporate one deuterium from NADPH-²H (but not from ²H₂O, as expected) into cinnamyl alcohol product and can incorporate deuterium in the presence of QD.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Terms such as “connected,” “attached,” “linked,” and “conjugated” are used interchangeably herein and encompass direct as well as indirect connection, attachment, linkage or conjugation unless the context clearly dictates otherwise.

Where a range of values is recited, it is to be understood that each intervening integer value, and each fraction thereof, between the recited upper and lower limits of that range is also specifically disclosed, along with each sub-range between such values. The upper and lower limits of any range can independently be included in or excluded from the range, and each range where either, neither or both limits are included is also encompassed. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each sub-combination of the elements of that combination is also specifically disclosed and is within the scope of the invention. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

The terms “semiconductor nanocrystal,” “SCNC,” “SCNC nanocrystal,” “quantum dot,” and “QD” are used interchangeably herein and refer to an inorganic crystallite of about 1 nm or more and about 1000 nm or less in diameter or any integer or fraction of an integer therebetween, preferably at least about 2 nm and about 50 nm or less in diameter or any integer or fraction of an integer therebetween, more preferably at least about 2 nm and about 20 nm or less in diameter (for example about 2, 3, 4, 5, 6, 7, 8, 9, 10, 11, 12, 13, 14, 15, 16, 17, 18, 19, or 20 nm). A given QD sample will have a range of sizes that is characterized by a low range of dispersity or a range of low polydispersity.

A QD is capable of emitting electromagnetic radiation upon excitation (i.e., the QD is luminescent) and includes a “core” of one or more first semiconductor materials, and may be surrounded by a “shell” of a second semiconductor material. A QD core surrounded by a semiconductor shell is referred to as a “core/shell” QD. The surrounding “shell” material will preferably have a bandgap energy that is larger than the bandgap energy of the core material and may be chosen to have an atomic spacing close to that of the “core” substrate.

The core and/or the shell can be a semiconductor material including, but not limited to, those of the groups II-VI (ZnS, ZnSe, ZnTe, US, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like) materials, PbSe, and an alloy or a mixture thereof. Preferred shell materials include ZnS.

A QD is optionally surrounded by a “coat” of an organic capping agent. The organic capping agent may be any number of materials, but has an affinity for the QD surface. In general, the capping agent can be an isolated organic molecule, a polymer (or a monomer for a polymerization reaction), an inorganic complex, or an extended crystalline structure. The coat can be used to convey solubility, e.g., the ability to disperse a coated QD homogeneously into a chosen solvent, functionality, binding properties, or the like. In addition, the coat can be used to tailor the optical properties of the QD.

Thus, the quantum dots herein include a coated core, as well as a core/shell QD.

The term “nanoparticle” as used herein includes the above-mentioned QDs in addition to other nano-scale and smaller particles such as carbon nanotubes, proteins, polymers, dendrimers, viruses, and drugs. A nanoparticle has a size of less than about 1 micron, optionally less than about 900, 800, 700, 600, 500, 400, 300, 100, 80, 60, 50, 40, 30, 20, 10, 9, 8, 7, 6, 5, 4, 3, 2, or 1 nanometers. A nanoparticle may have various shapes such as a rod, a tube, a sphere, and the like. Nanoparticles may be made from various materials including metals, carbon (such as carbon nanotubes), polymers, and combinations thereof. A nanoparticle for cytosolic delivery by a peptide may be referred to as a cargo or payload.

Overview

Incorporating stable isotopes into small molecules is an important synthetic process for creating new chemical building blocks with applications in chemistry and medicine. For example, in medical applications one can label and follow a drug candidate to determine how it is metabolized and confirm its mechanism of action. Here, two exemplary multi-enzyme systems were used to demonstrate the incorporation of a prototypical stable isotope, deuterium, into reaction intermediates and the final product in a controlled and site-specific manner. Two different deuterium sources were employed, solvent and cofactor, to follow the incorporation of stable isotopes at site-specific locations in the target molecule. By relying on these external sources for isotopes and on multi-enzyme assistance for incorporation, one can reliably incorporate stable isotopes in a green fashion, with little to no hazardous waste generation.

In this case, quantum dots serve as prototypical nanoparticles to assemble enzymes into functional nanoclusters through metal affinity binding, for example between a histidine tag on the enzyme interacting with a Zn-rich surface of a quantum dot. These nanoparticles serve to stabilize the tertiary structure of the enzyme, and when different enzymes are assembled on the nanoparticle, to facilitate “channeling” of their catalytic processes. Channeling is an enzymatic phenomenon where the product of one enzyme is passed to the next enzyme in the catalytic cascade that is also placed proximal to it and thus overcomes diffusion limitations and increases the overall catalytic efficiency or flux of the multistep enzymatic system. Nanoparticles have been shown to increase enzymatic efficiency under specific reaction regimes allowing for more product production with less enzyme. See, for example, U.S. Patent Application Publication No. 2018/0171325 and Vranish et al., ACS Nano 2018, 12, 8, 7911-7926.

### EXAMPLES

Enzymes were assembled to 525 nm emitting QDs via metal affinity coordination between terminal histidine tags present in the enzymes attaching to the Zn-rich surfaces of the QDs. Binding was confirmed by changes in electrophoretic mobility shifts seen on agarose gels.

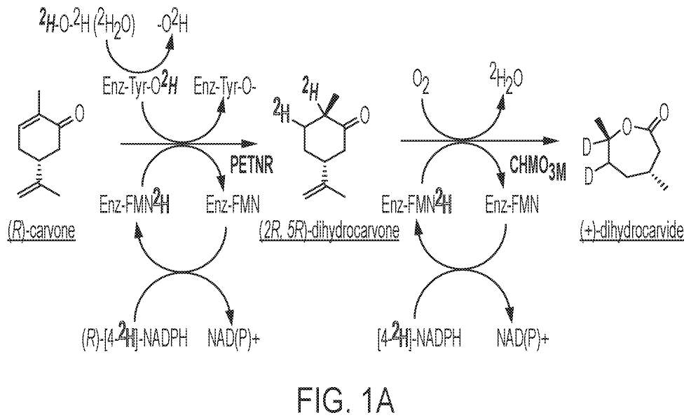

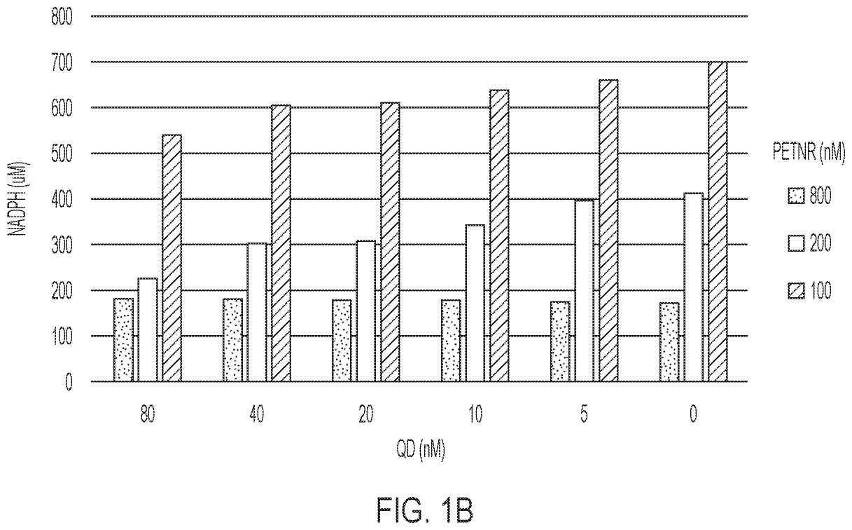

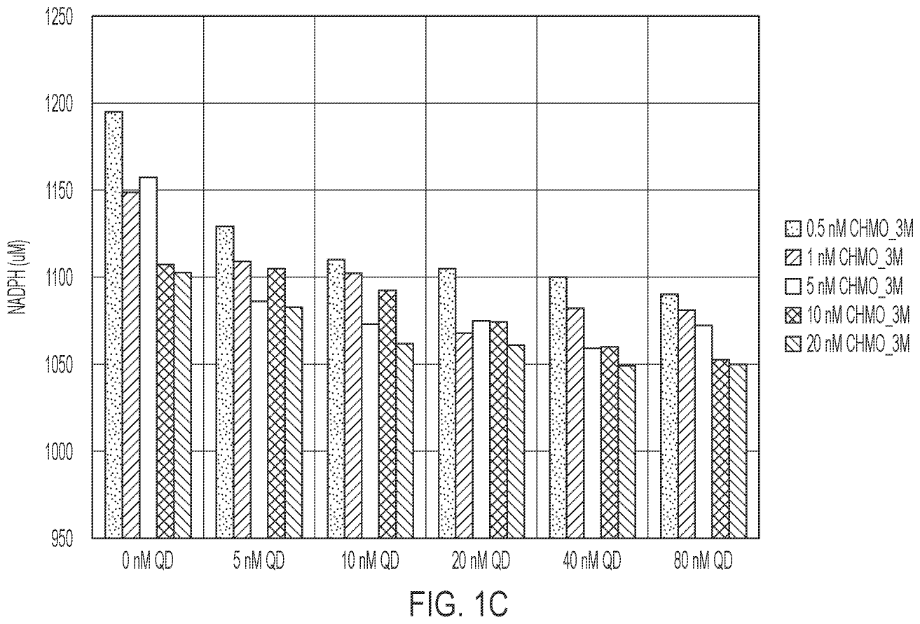

FIG. 1A illustrates a first exemplary reaction scheme involved the incorporation of deuterium into (+)-dihydrocarvide using a cascade involving the enzymes (a) pentaerythritol tetranitrate reductase (PETNR) and (b) flavin-dependent cyclohexanone monooxygenase triple variant F249A/F280A/F435A (CHMO_{3M}). In FIG. 1A, the typeface style (either bold italics or outline) is used to indicate which species contribute which deuterium atoms (alternately depicted as “²H” or “D”), and where they are incorporated into the (+)-dihydrocarvide. The deuterium atoms depicted in bold italic formatted text come from deuterated water while the deuterium in outline text comes from deuterated nicotinamide adenine dinucleotide phosphate (NADPH). FIG. 1B shows results from determining the activity of (PETNR) on and off QDs at various concentration of enzyme and QD. As the amount of enzyme increases and/or the amount of QD increases, the amount of NADPH decreases indicating the reaction is proceeding and enhanced when attached to a QD. FIG. 1C shows results from determining the activity of CHMO_{3M} on and off QDs at various concentration of enzyme and QD. As with PETNR, as the amount of enzyme increases and/or the amount of QD increases, the amount of NADPH decreases indicating the reaction is proceeding and enhanced when attached to a QD.

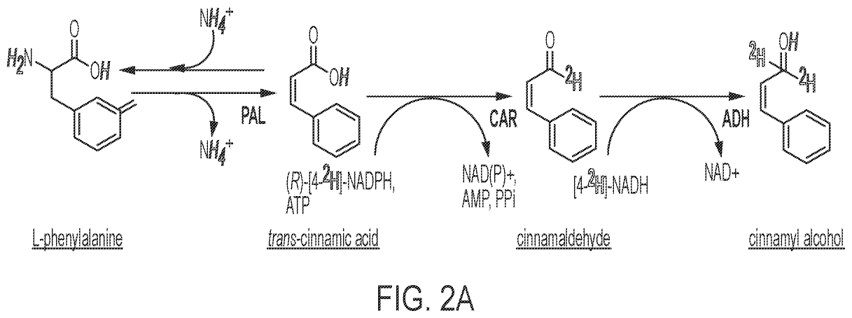

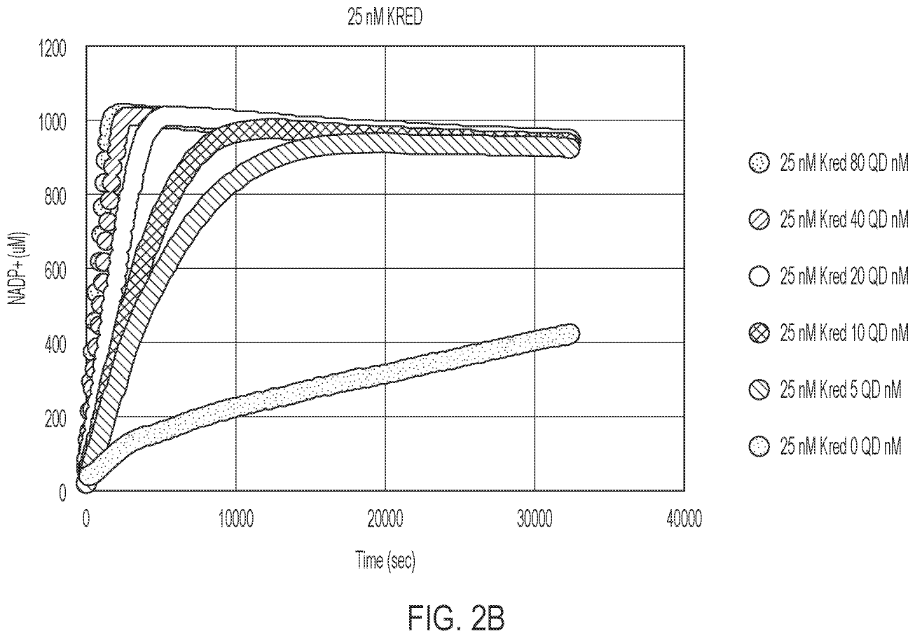

FIG. 2A shows a second exemplary reaction scheme incorporated deuterium into cinnamyl alcohol using a cascade of the enzymes (a) phenylalanine ammonia lyase (PAL), (b) carboxylic acid reductase (CAR), and (c) alcohol dehydrogenase (ADH, also referred to as keto-reductase or KRED). Differing typefaces indicate where deuterium can be incorporated into each small molecule and the hydrogens that are likely exchanged for deuterium in D₂O. FIG. 2B shows results of KRED converting NADPH to NADP+either free in solution or as assembled to increasing concentrations of QD. As the concentrations increases in the presence of KRED, the rate of KRED to convert NADPH to NADP+increases.

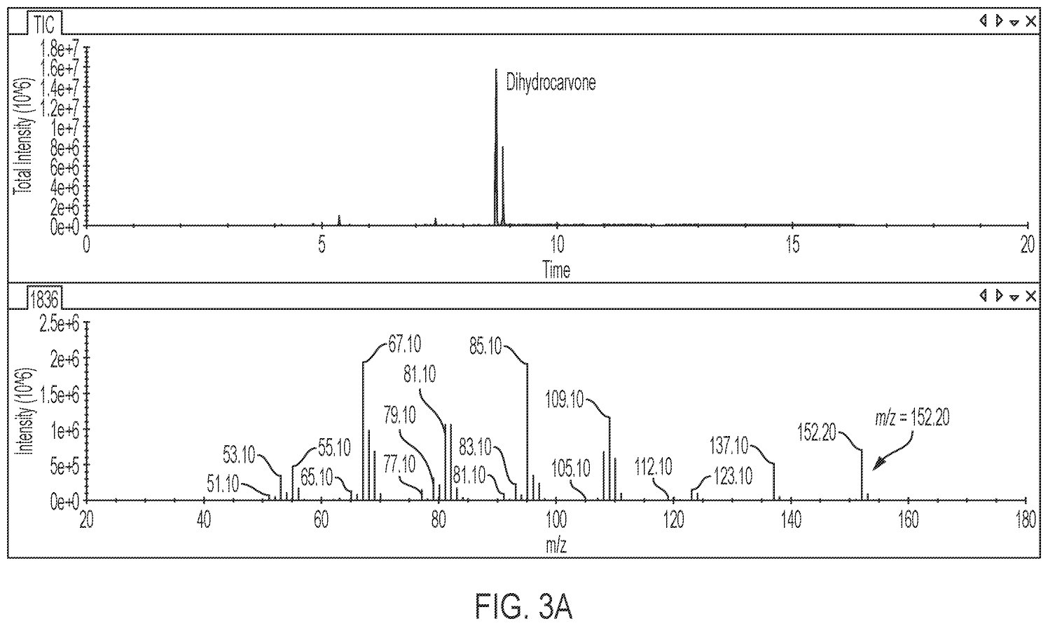

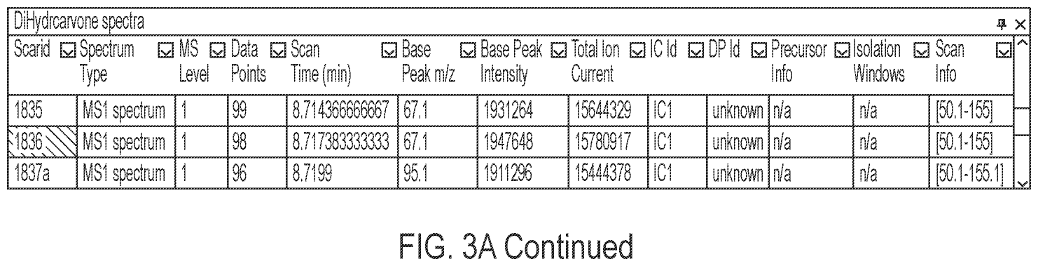

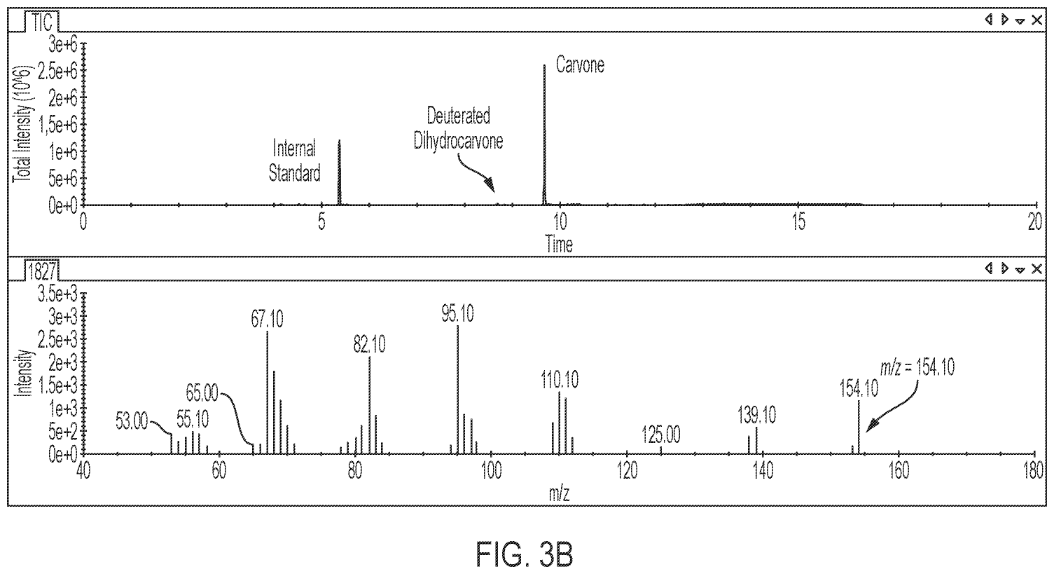

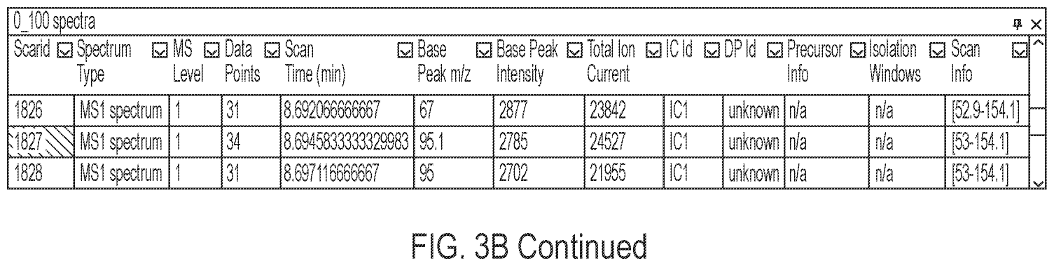

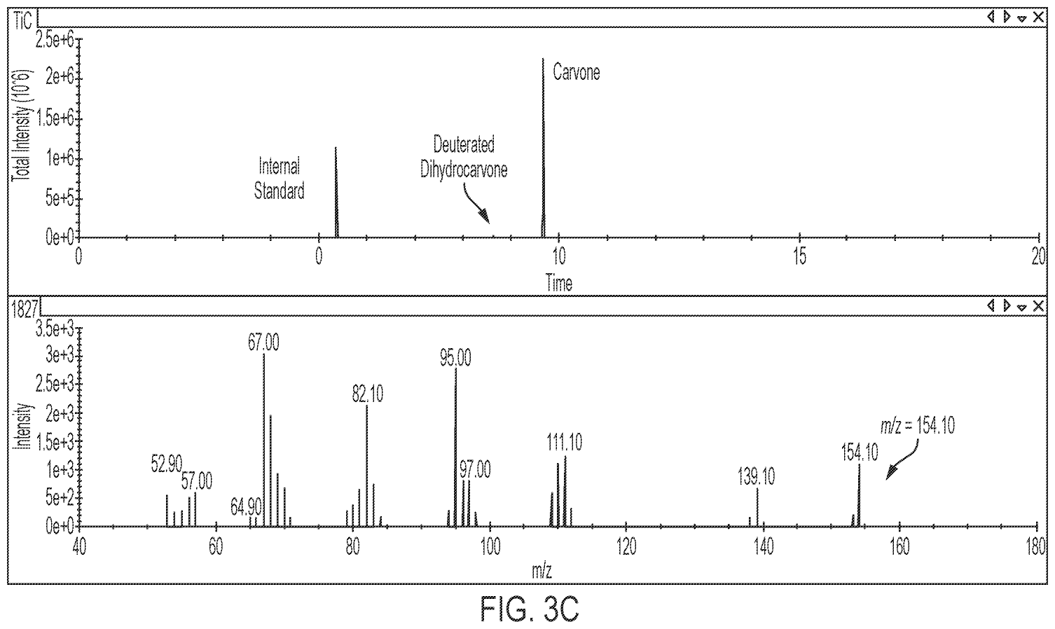

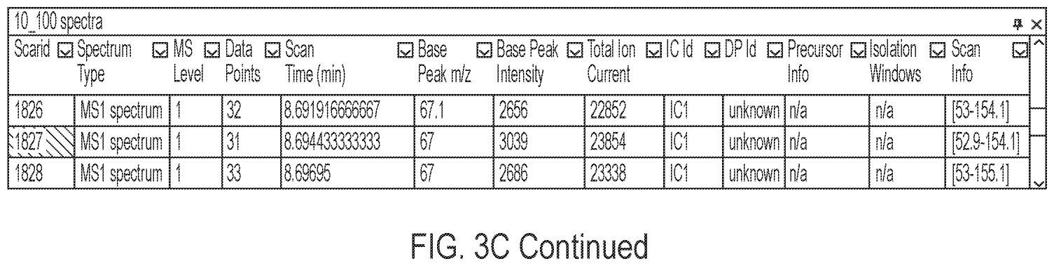

FIGS. 3A-3C provide GC/MS spectra indicating PETNR can incorporate one deuterium from ²H₂O and one deuterium from NADPH-²H into dihydrocarvone product and can incorporate deuterium in the presence of QD. FIG. 3A shows GC/MS spectra of a dihydrocarvone standard with the approximately-expected mass to charge ratio (m/z) of 152.2. FIG. 3B shows GC/MS spectra of reaction of PETNR without QD in ²H₂O and NADPH-²H solution producing doubly-deuterated dihydrocarvone with the approximately-expected m/z of 154.1. FIG. 3C shows GC/MS spectra of reaction of PETNR with QD in ²H₂O and NADPH-²H solution producing doubly-deuterated dihydrocarvone with the approximately-expected m/z of 154.1.

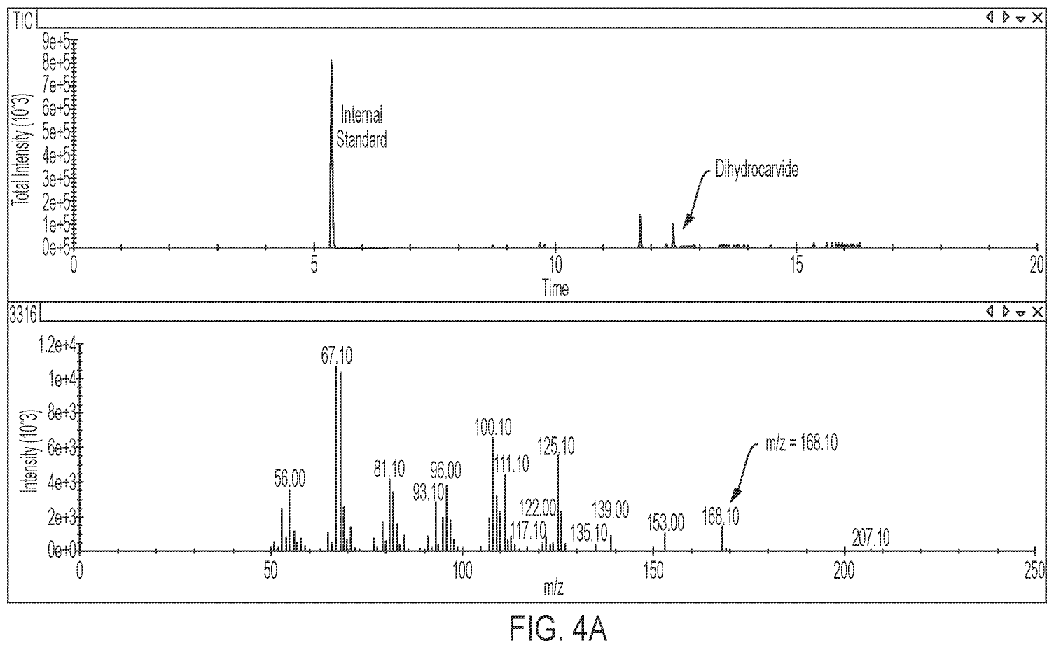

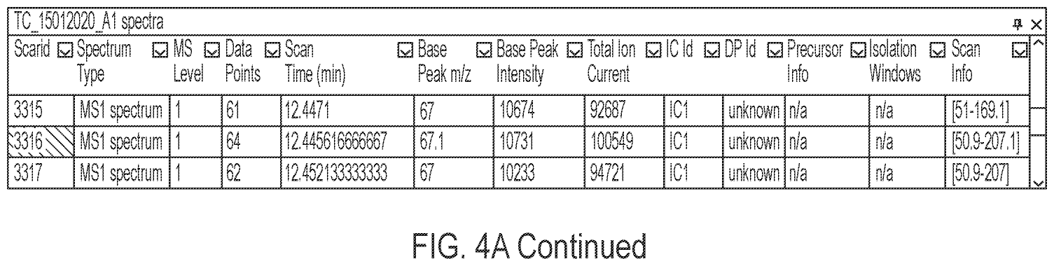

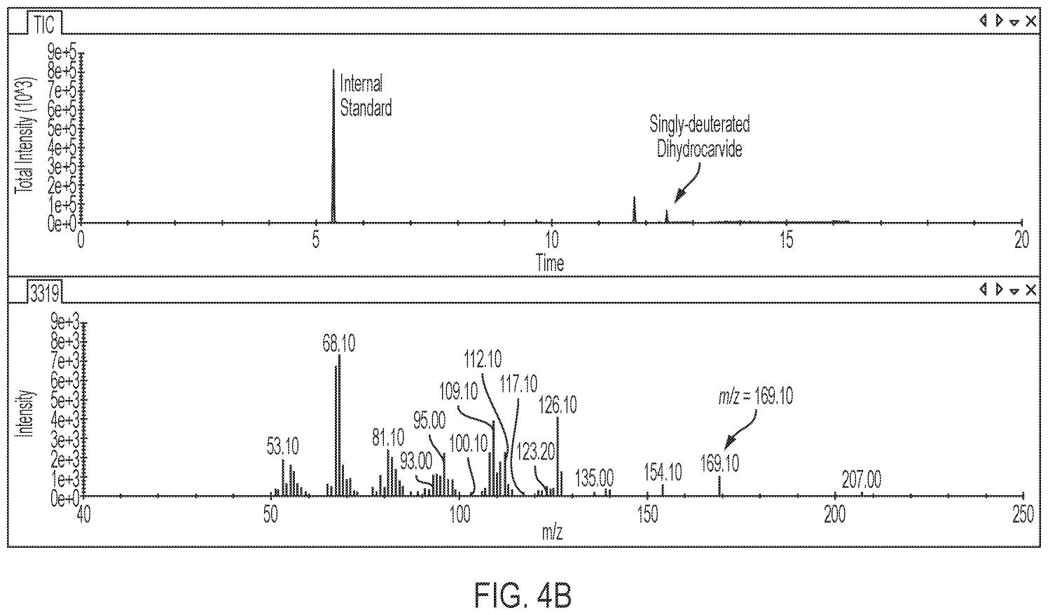

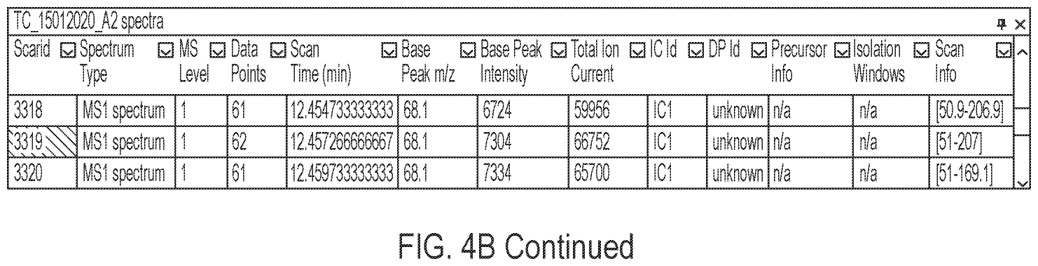

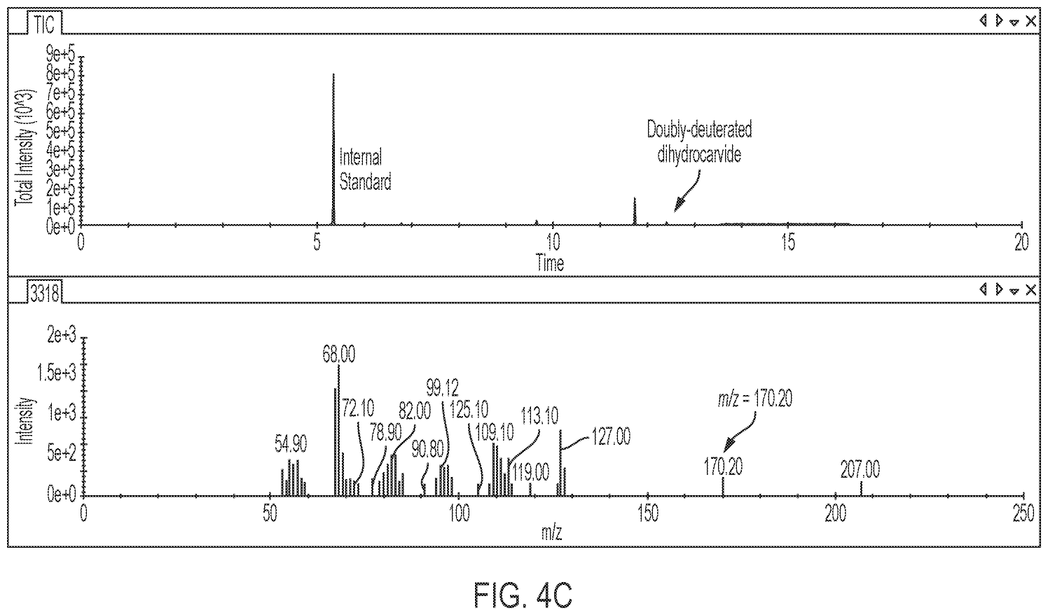

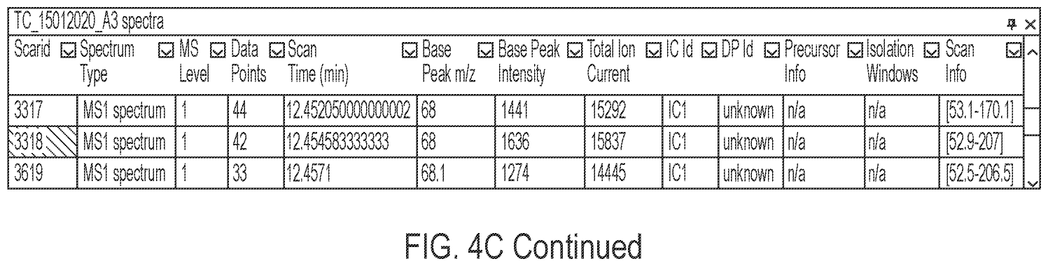

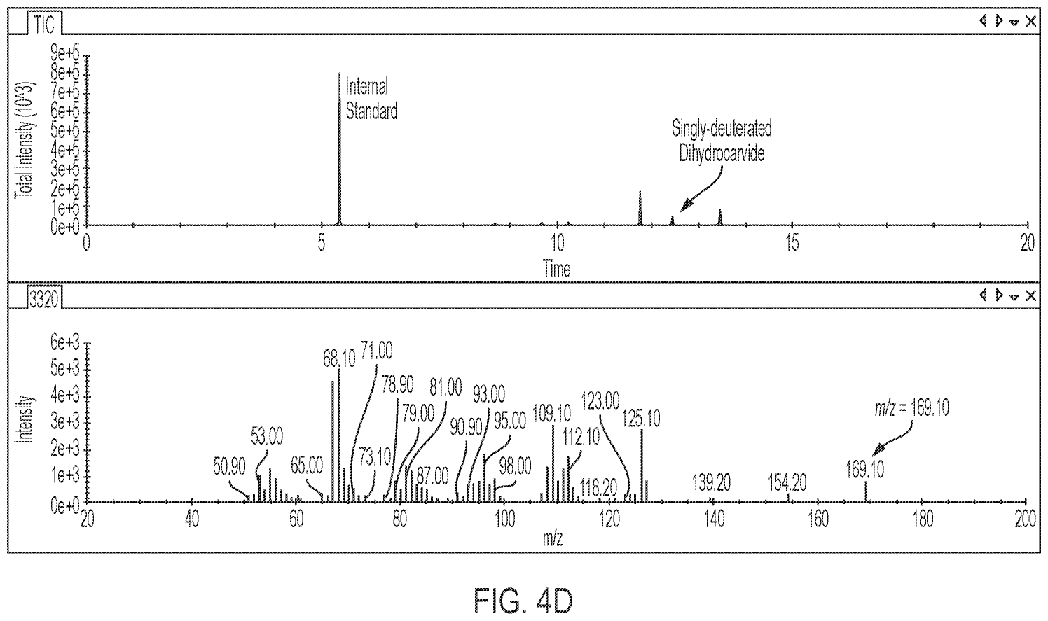

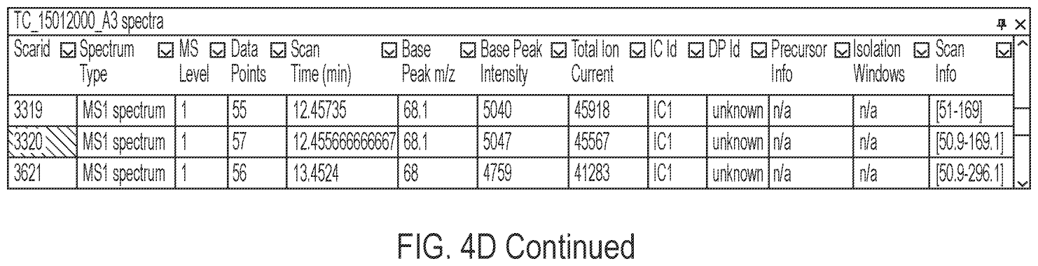

FIGS. 4A-4D are GC/MS spectra indicating PETNR and CHMO_{3M} can incorporate one deuterium from ²H₂O and one deuterium from NADPH-²H into dihydrocarvide product, and can incorporate deuterium in the presence of QD. FIG. 4A is GC/MS spectra of reaction of PETNR and CHMO_{3M} without QD in H₂O and NADPH solution producing dihydrocarvide with the approximately-expected m/z of 168.1. FIG. 4B shows GC/MS spectra of reaction of PETNR and CHMO_{3M} without QD in ²H₂O and NADPH solution producing singly-deuterated dihydrocarvide with the approximately-expected m/z of 169.1. FIG. 4C is GC/MS spectra of reaction of PETNR and CHMO_{3M} without QD in ²H₂O and NADPH-²H solution producing doubly-deuterated dihydrocarvide with the approximately-expected m/z of 170.2. FIG. 4D shows GC/MS spectra of reaction of PETNR and CHMO_{3M} with QD in ²H₂O and NADPH solution producing singly-deuterated dihydrocarvide with the approximately-expected m/z of 169.1

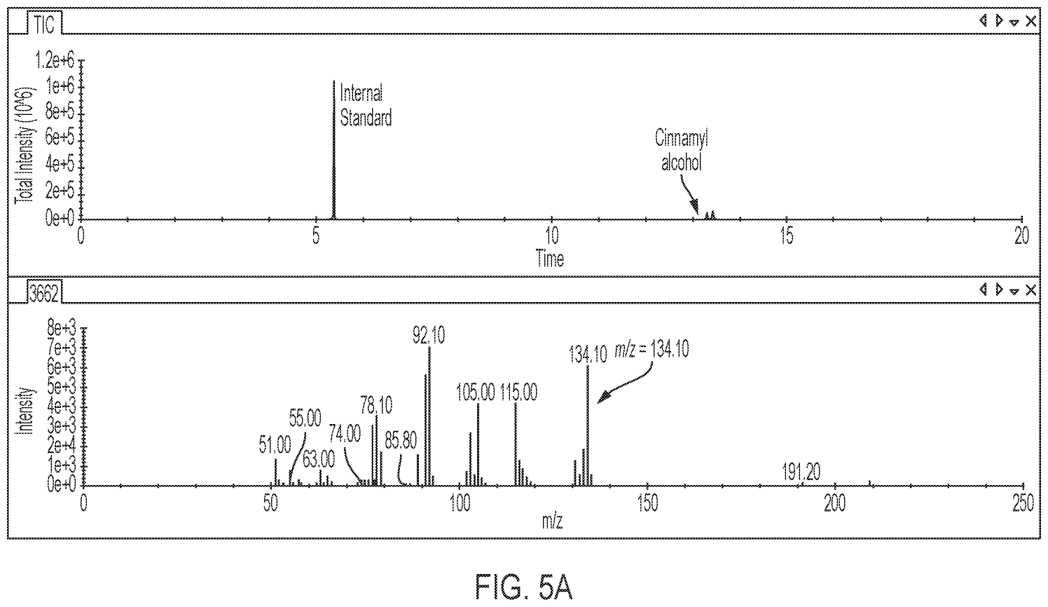

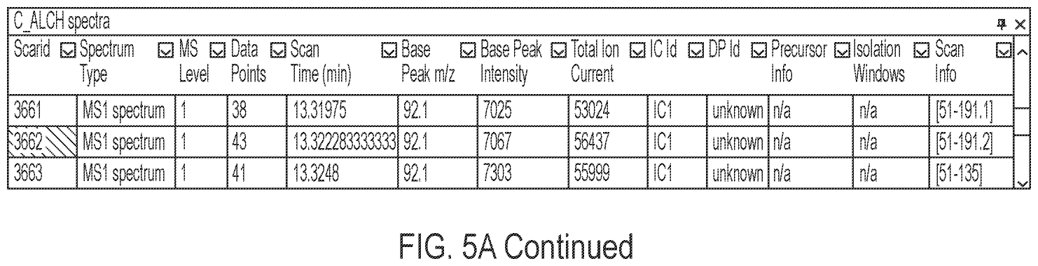

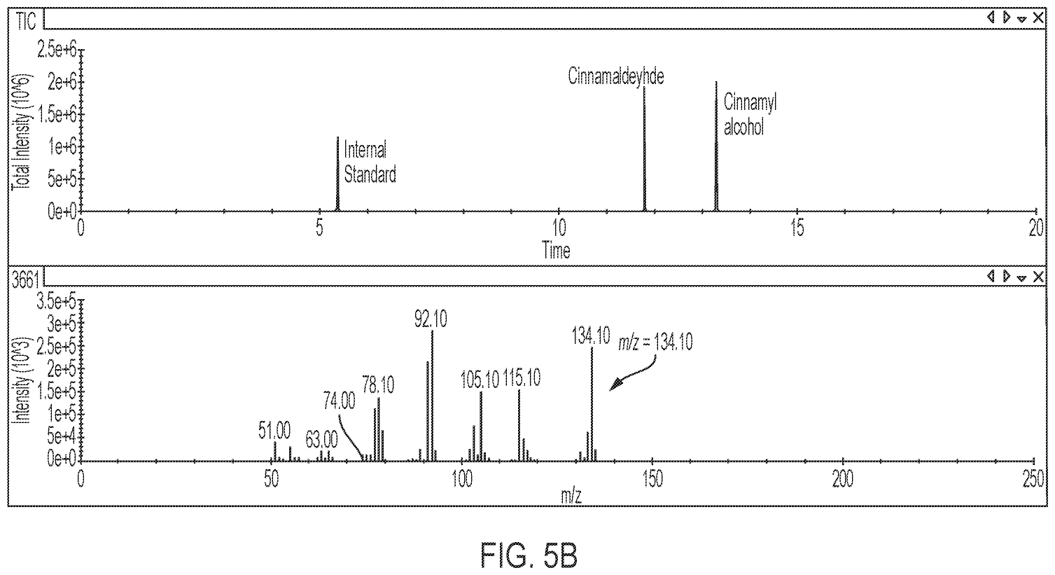

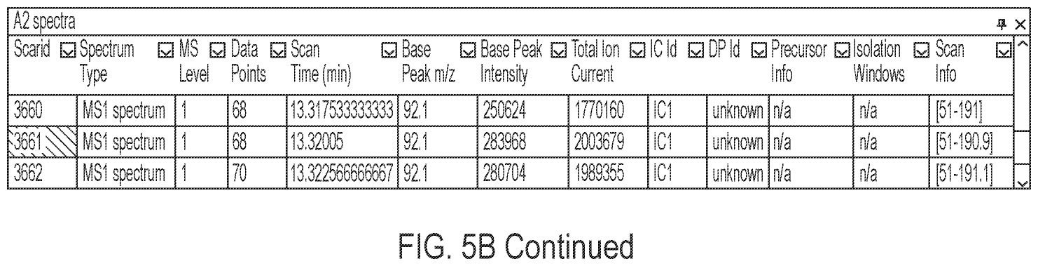

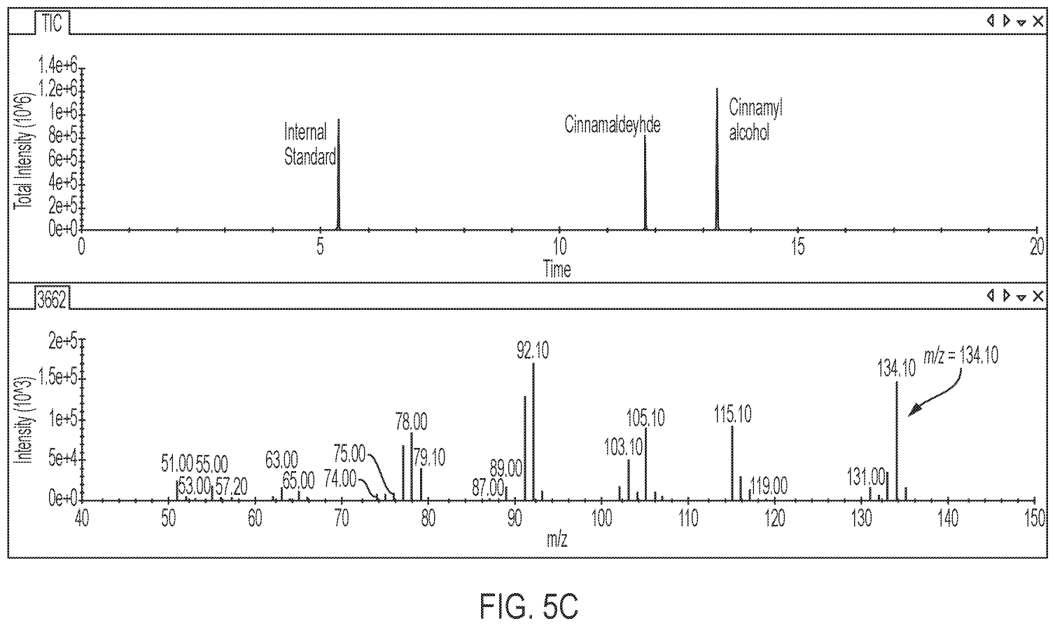

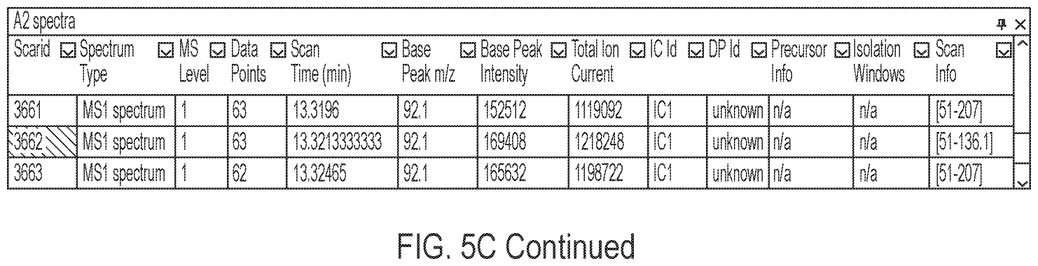

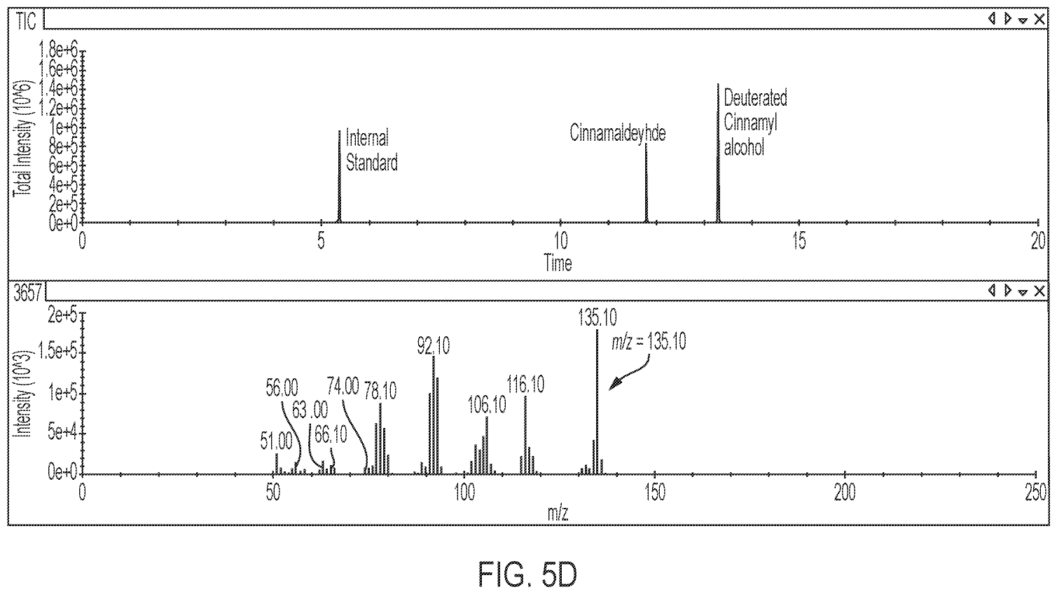

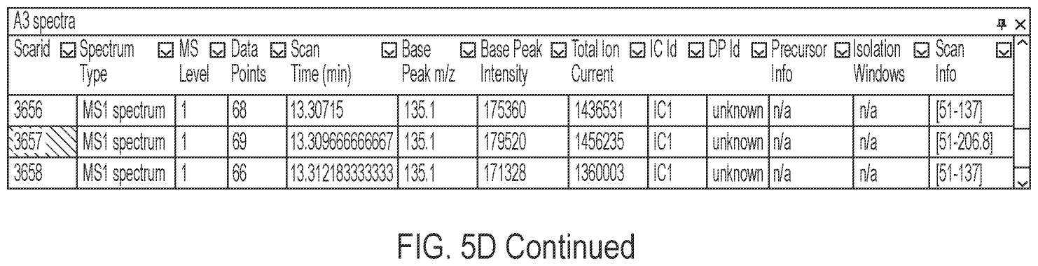

FIGS. 5A-5D provide GC/MS spectra indicating KRED can incorporate one deuterium from NADPH-²H (but not from ²H₂O, as expected) into cinnamyl alcohol product and can incorporate deuterium in the presence of QD. FIG. 5A is GC/MS spectra of cinnamyl alcohol standard with the approximately-expected m/z of 134.1. FIG. 5B shows GC/MS spectra of reaction of KRED in H₂O and NADPH solution producing cinnamyl alcohol with the approximately-expected m/z of 134.1. FIG. 5C is GC/MS spectra of reaction of KRED in ²H₂O and NADPH solution producing cinnamyl alcohol with the approximately-expected m/z of 134.1. FIG. 5D is GC/MS spectra of reaction of KRED in ²H₂O and NADPH-²H solution producing singly-deuterated cinnamyl alcohol with the approximately-expected m/z of 135.1.

### Further Embodiments

Other materials, such as DNA nanostructures, nanoplatelets, and gold nanoparticles might be used for enzyme or substrate immobilization.

In addition to deuterium as used in the examples, this technique can be adapted for the incorporation of other radioisotopes into compounds. For example, ¹⁸F-labeled compounds might be prepared, which can be useful for medical imaging.

Advantages

Using enzymes as described herein to incorporate stable isotopes in a controlled and site specific manner offers a number of advantages.

The technique provides an alternative to traditional chemistry to incorporate stable or radioactive isotopes such as deuterium, ¹⁵N, or another isotope into small molecules in a controlled and site specific fashion. This novel green chemistry occurs under benign conditions minimizing chemical waste. Furthermore, building blocks containing stable or radioactive isotopes are suitable for further chemical modification.

QDs and other NPs can stabilize these enzymes, preventing their denaturation, allowing for efficient isotope incorporation. QDs and other NPs can host multiple enzymes on their surface through a variety of self-assembly techniques such as metal affinity binding. Assembling multiple versions of different enzymes associated with a chemical reaction onto a NP or QD can improve the overall efficiency of reaction through enzyme stabilization and substrate channeling. Luminescent QDs can be easily functionalized with a wide variety of surface ligands that provide different surface charges, polarities, and steric bulk, that can influence the reaction of the enzymes that are displayed on their surface. Both eukaryotic and prokaryotic enzymes can be incorporated into the same cascaded reaction. The choice of enzymes and reactions specifies the order in which the incorporations are made. The technique can utilize both anabolic and catabolic enzymatic pathway and is amenable to chemoenzymatic approaches as well as the use of engineered and modified enzymes which may not work in cellular environments.

A further advantage is an increased rates of reaction in producing isotope-labeled compounds as compared to prior art techniques. Accordingly, because of a relatively quicker incorporation of radioisotopes (that decay over time), the resulting compound has more active radioisotope when used, or, along the same lines, in the cases of unstable compounds, less degradation of the compound occurs.

Moreover, the use of enzymatic synthesis often provides better stereo/regio-chemistry of incorporation.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Algar, W. R., T. Jeen, M. Massey, W. J. Peveler and J. Asselin (2019). “Small Surface, Big Effects, and Big Challenges: Toward Understanding Enzymatic Activity at the Inorganic Nanoparticle-Substrate Interface.” Langmuir 35(22): 7067-7091.
2. Blanco-Canosa, J. B., M. Wu, K. Susumu, E. Petryayeva, T. L. Jennings, P. E. Dawson, W. R. Algar and I. L. Medintz (2014). “Recent progress in the bioconjugation of quantum dots.” Coordination Chemistry Reviews 263: 101-137.
3. Breger, J. C., E. Oh, K. Susumu, W. P. Klein, S. A. Walper, M. G. Ancona and I. L. Medintz (2019). “Nanoparticle Size Influences Localized Enzymatic Enhancement-A Case Study with Phosphotriesterase.” Bioconjugate Chemistry 30(7): 2060-2074.
4. Diaz, S. A., J. C. Breger and I. L. Medintz (2016). Monitoring Enzymatic Proteolysis Using Either Enzyme- or Substrate-Bioconjugated Quantum Dots. Rational Design of Enzyme Nanomaterials. C. V. Kumar. 571: 19-54.
5. Prasuhn, D. E., J. R. Deschamps, K. Susumu, M. H. Stewart, K. Boeneman, J. B. Blanco-Canosa, P. E. Dawson and I. L. Medintz (2010). “Polyvalent Display and Packing of Peptides and Proteins on Semiconductor Quantum Dots: Predicted Versus Experimental Results.” Small 6(4): 555-564.
6. Susumu, K., L. D. Field, E. Oh, M. Hunt, J. B. Delehanty, V. Palomo, P. E. Dawson, A. L. Huston and I. L. Medintz (2017). “Purple-, Blue-, and Green-Emitting Multishell Alloyed Quantum Dots: Synthesis, Characterization, and Application for Ratiometric Extracellular pH Sensing.” Chemistry ofMaterials29(17): 7330-7344.
7. Susumu, K., E. Oh, J. B. Delehanty, J. B. Blanco-Canosa, B. J. Johnson, V. Jain, W. J. Hervey, W. R. Algar, K. Boeneman, P. E. Dawson and I. L. Medintz (2011). “Multifunctional Compact Zwitterionic Ligands for Preparing Robust Biocompatible Semiconductor Quantum Dots and Gold Nanoparticles.” Journal of the American Chemical Society 133(24): 9480-9496.
8. Vranish J. N., Ancona M. G, Walper S. A., and M—dintz I. L. (2018). “Pursuing the Promise of Enzymatic Enhancement with Nanoparticle Assemblies”. Langmuir 34 (9): 2901-2925.
9. Klumbys, E., Zebec, Z., Weise N. J., Turner N. J., and Scrutton N. S. (2019). Bio-derived Production of Cinnamyl Alcohol via a Three Step Biocatalytic Cascade and Metabolic Engineering. Green Chem. 20(3): 658-663.
10. Ascue Avalos G. A., Toogood H. S., Tait S., Messiha H. L., Scrutton N. S. (2019). From Bugs to Bioplastics: Total (+)-Dihydrocarvide Biosynthesis by Engineered Escherichia coli. ChemBioChem 20: 785-792.
11. Vranish, J. N, Ancona, M. G., Oh, E., Susumu, K., Lasarte Aragones, G., Breger, J. C., Walper, S. A., and Medintz, I. L. (2018) Enhancing Coupled Enzymatic Activity by Colocalization on Nanoparticle Surfaces: Kinetic Evidence for Directed Channeling of Intermediates. ACS Nano 12: 7911-7926.

What is claimed is:

1. A method of incorporating a radioisotope into a product molecule comprises:
providing a nanoparticle attached to a plurality of enzymes configured as an enzymatic cascade such that a product of a first enzyme is a substrate of the second enzyme and so forth;
providing a radioisotope source and a source substrate to the enzymatic cascade; and
allowing the enzymatic cascade to act on the radioisotope source and the source substrate, thereby transferring a radioisotope from the radioisotope source into a product molecule.

2. The method of claim 1, wherein the radioisotope source is provided in the form of a solvent and/or one or more cofactors of the enzymes.

3. The method of claim 1, wherein the radioisotope source is a deuterium source.

4. The method of claim 3, wherein the deuterium source is selected from the group consisting of as deuterated water, deuterated nicotinamide adenine dinucleotide phosphate (NADH-²11), deuterated reduced nicotinamide adenine dinucleotide (NADH-²11), and combinations thereof.

5. The method of claim 1, wherein the enzymatic cascade comprises one or more reductase enzymes effective to utilize at least one of the radioisotope sources.

6. The method of claim 5, wherein said one or more reductase enzymes comprise pentaerythritol tetranitrate reductase (PETNR) and/or carboxylic acid reductase (CAR).

7. The method of claim 1, wherein
the enzymatic cascade comprises pentaerythritol tetranitrate reductase and a cyclohexanone monooxygenase;
and the product molecule is dihydrocarvide.

8. The method of claim 1, wherein
the enzymatic cascade comprises phenylalanine ammonia lyase (PAL), carboxylic acid reductase (CAR), and alcohol dehydrogenase; and
the product molecule is cinnamyl alcohol.

9. A method of incorporating deuterium into (+)-dihydrocarvide comprising:
providing a nanoparticle attached to two enzymes, namely (a) pentaerythritol tetranitrate reductase (PETNR) and (b) flavin-dependent cyclohexanone monooxygenase (CHMO);
contacting the nanoparticle with carvone and one or more deuterium sources; and
allowing the enzymes to act on the carvone and the one or more deuterium sources, thereby producing deuterated dihydrocarvide,
wherein the one or more deuterium sources comprise deuterated water. deuterated nicotinamide adenine dinucleotide phosphate, or a combination thereof.

10. The method of claim 9, wherein the CHMO is the variant F249A/F280A/F435A (CHMO_{3M}).

11. The method of claim 9, wherein said deuterium sources comprise both deuterated water and deuterated nicotinamide adenine dinucleotide phosphate.

12. A method of incorporating deuterium into cinnamyl alcohol comprising:
providing a nanoparticle attached to three enzymes, namely (a) phenylalanine ammonia lyase (PAL), (b) carboxylic acid reductase (CAR), and (c) alcohol dehydrogenase (ADH);
contacting the nanoparticle with phenylalanine and one or more deuterium sources; and
allowing the enzymes to act on the phenylalanine and the one or more deuterium sources, thereby producing deuterated cinnamyl alcohol, wherein
the one or more deuterium sources comprise deuterated nicotinamide adenine dinucleotide phosphate, deuterated reduced nicotinamide adenine dinucleotide, or a combination thereof.

13. The method of claim 12, wherein said deuterium sources comprise both deuterated nicotinamide adenine dinucleotide phosphate and deuterated reduced nicotinamide adenine dinucleotide.
